(19) United States
(12) Patent Application Publication
(10) Pub. No.: US 2014/0228925 A1
(43) Pub. Date: Aug. 14, 2014

(54) IMPLANTABLE MEDICAL LEAD WITH BLOOD SEAL

(71) Applicant: Johan Eckerdal, Knivsta (SE)

(72) Inventor: Johan Eckerdal, Knivsta (SE)

(73) Assignee: ST. JUDE MEDICAL AB, Jarfalla (SE)

(21) Appl. No.: 13/641,654

(22) PCT Filed: Oct. 27, 2011
(86) PCT No.: PCT/EP11/68857

§ 371 Date: Oct. 16, 2012

(51) Int. Cl. A61N 1/05

(52) U.S. Cl. CPC A61N 1/0573
USPC 607/121; 607/127

(57) ABSTRACT

An implantable medical lead comprises an insulating tubing with a channel housing a conductor electrically connected to a shaft present in a lumen of a bearing. The shaft is also connected to a fixation helix present in a lumen of a lead header. A clot-inducing structure of a thrombogenic material is present in a distal portion of the channel and/or in the lumen of the bearing to trigger formation of a clot when blood enters the implantable medical lead and thereby inhibit blood from entering further into the channel.

### TECHNICAL FIELD

The present embodiments generally relate to implantable medical leads, and in particular implantable medical leads with a blood seal

### BACKGROUND

Various types of body-implantable leads are known and used in the medical field. For example, implantable medical devices (IMDs), such as pacemakers, cardiac defibrillators and cardioverters, are in operation connected to implantable medical leads for sensing cardiac function and other diagnostic parameters and delivering stimulation pulses. For example, endocardial leads are attached at their proximal end to an IMD and at their distal end to the endocardium of a cardiac chamber.

Implantable medical leads can generally be divided into two groups depending on how they are anchored at a target site in the patient body. So called passive fixation leads comprise structures in connection with their distal end, such as fins, tines or collars. The implantable medical lead is then attached to a target site by these structures that become entangled in connective tissue and thereby anchor the implantable medical lead to the tissue. The other group comprises so called active fixation leads. Such leads are not dependent on passive growth of connective tissue to anchor the implantable medical lead. In clear contrast, an active fixation lead comprises a fixation helix that is movable out from the lead body and can thereby be screwed into a target tissue in the patient body.

Active fixation leads generally lead to a more reliable lead attachment but this improvement comes with design challenges of the implantable medical leads. Thus, since the fixation helix is to be extendable relative to the lead body, the most distal end of the implantable medical lead comprises an opening through which the fixation helix is screwed. However, this opening enables blood present outside of the implantable medical lead in the patient body to enter into the lead structure. There the blood will coagulate and form one or more clots somewhere along the lead. Such clots may, however, interfere with the operation of a stylet that is generally inserted into the lead during implantation and explantation operations. In addition, blood clots can impair the helix function of the implantable medical lead.

In the art, various blood seals have been proposed and which are arranged in the implantable medical lead to prevent blood from reaching further into the inside of the lead. However, such blood seals are difficult to design in order to be effective in preventing blood entry while at the same time allowing smooth helix extension and retraction. Thus, a common problem with known blood seals is either that the seal is too tight so that it becomes very hard to screw out and in the fixation helix or that the seal is not tight enough so that blood thereby passes the blood seal.

US 2002/0016622 discloses a blood seal for implantable medical leads of the so called active fixation type. The blood seal is in the form of an expandable hydrogel matrix arranged around a piston to which the fixation helix is attached or onto the fixation helix itself. Upon contact with blood entering into the implantable medical lead the hydrogel seal expands and will therefore fill up and seal off the gap between the piston or helix and the lead housing.

### SUMMARY

It is a general objective to provide an effective blood seal in an implantable medical device.

This and other objectives are met by embodiments disclosed herein.

Briefly, the embodiments relate to an implantable medical lead comprising a conductor at least partly present in a channel of an insulating tubing. The conductor has a first end electrically connected to a second end of a shaft at least partly present in a lumen of a bearing. The first end of the shaft is mechanically connected to a first end of a fixation helix at least partly present in a lumen of a lead header. A clot-inducing structure of at least one thrombogenic material is present in a distal portion of the channel and/or in the lumen of the bearing. The clot-inducing structure triggers formation of a blood clot when blood enters the implantable medical lead. The formed blood clot provides an effective blood seal and inhibits blood from entering further into the channel and towards an opposite proximal portion of the channel.

### BRIEF DESCRIPTION OF THE DRAWINGS

The invention, together with further objects and advantages thereof, may best be understood by making reference to the following description taken together with the accompanying drawings, in which:

FIG. 1 is a schematic overview of a human patient having an implantable medical device connected to an implantable medical lead according to an embodiment;

FIG. 2 illustrates an implantable medical lead according to an embodiment connectable to an implantable medical device;

FIG. 3 is a cross-sectional view of a distal portion of an implantable medical lead according to an embodiment;

FIG. 4 is a cross-sectional view of a distal portion of an implantable medical lead according to another embodiment;

FIG. 5 is a cross-sectional view of a distal portion of an implantable medical lead according to a further embodiment;

FIG. 6 is a cross-sectional view of a distal portion of an implantable medical lead according to yet another embodiment;

FIG. 7 is a cross-sectional view of a distal portion of an implantable medical lead according to a further another embodiment;

FIG. 8 is a cross-sectional view of a distal portion of an implantable medical lead according to another embodiment;

FIG. 9 is a cross-sectional view of a distal portion of an implantable medical lead according to yet another embodiment;

FIG. 10 is a cross-sectional view of a distal portion of an implantable medical lead according to another embodiment;

FIG. 11 is a cross-sectional view of a distal portion of an implantable medical lead according to yet another embodiment; and

FIG. 12 is cross-sectional view of a cage with a mesh of thrombogenic material that can be used in an implantable medical device according to an embodiment.

### DETAILED DESCRIPTION

Throughout the drawings, the same reference numbers are used for similar or corresponding elements.

The present embodiments generally relate to implantable medical leads, and in particular such leads equipped with a blood seal. Thus, the sealing functionality of the embodiments prevents blood from entering or at least reduces the amount of blood that can enter the implantable medical lead during and/or following implantation in a subject body, preferably a body of a mammalian subject and in particular of a human subject. The blood seal of the embodiments is designed to provide an effective sealing function while not interfering with the operation of a fixation helix of the implantable medical lead.

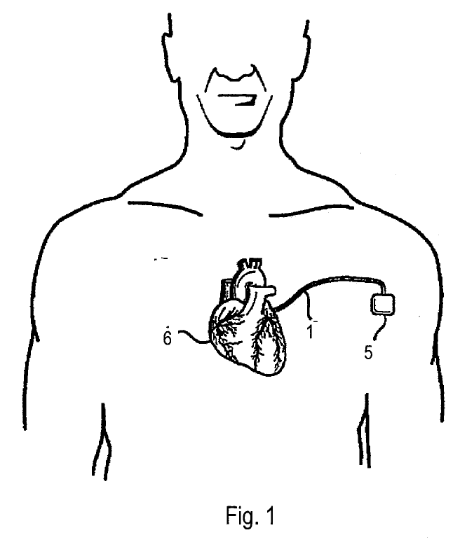

An implantable medical lead 1 is in operation, as is illustrated in FIG. 1, connected to an implantable medical device (IMD) 5, such as a pacemaker, defibrillator or cardioverter. The implantable medical lead 1 thereby provides the connection between the IMD 5 and the target tissue, such as a heart 6 of the subject. Thus, the IMD 5 is generally implanted remotely relative to the heart 6, in a so called device pocket. The implantable medical lead 1 then forms the electrical connection between the IMD 5 and the heart 6. The implantable medical lead 1 is configured to be at least partly implanted in or in connection with a ventricle or atrium of the heart 6.

However, the implantable medical leads 1 of the embodiments are not limited to be connectable to IMDs 5 designed for cardiac applications. Hence, the implantable medical leads 1 could instead be connectable to IMDs 5 in the form of neurological stimulators, physical signal recorders, etc.

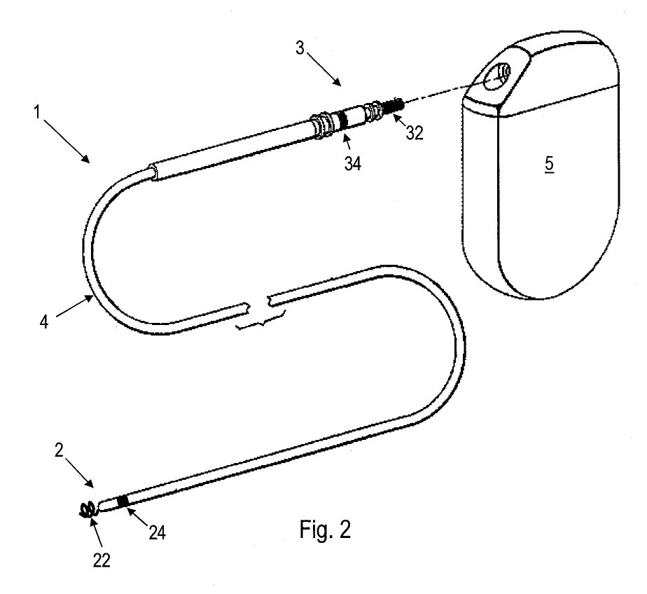

FIG. 2 is a schematic overview of an implantable medical lead 1 according to an embodiment. The implantable medical lead 1 comprises a distal end 2 designed to be introduced into a suitable pacing site to enable delivery of pacing pulses and sensing electric activity of the tissue, such as heart, at the particular pacing site. At least one electrode 22, 24, generally denoted pacing and sensing electrode in the art, is arranged in connection with the distal end 2. It is this or these electrode(s) 22, 24 that deliver(s) pacing pulses to the tissue and capture(s) electric signals originating from the tissue. Implantable medical leads comprising a single electrode are denoted unipolar leads in the art. FIG. 2 illustrates a so-called bipolar implantable medical lead 1 having two electrodes 22, 24 in connection with the distal end 2. The embodiments are, however, not limited to unipolar or bipolar leads but can also be applied to tripolar, quadropolar or other multipolar implantable medical leads having three, four or more electrodes, respectively.

The implantable medical lead 1 comprises a fixation helix 22 in its distal end 2. This fixation helix 22 is movable relative to a leady body 4 of the implantable medical lead 1 and can be extended out from the distal end 2 and be retracted into the implantable medical lead 1.

The fixation helix 22 generally constitutes one of the electrodes of the implantable medical lead 1.

An opposite or proximal end 3 of the implantable medical lead 1 is configured to be mechanically and electrically connected to an IMD 5. The proximal end 3 comprises at least one electrode terminal 32, 34 that provide the electric interface of the implantable medical lead 1 towards the IMD 5. Thus, each electrode terminal 32, 34 is connected to a respective connector terminal in the IMD 5 to thereby provide electric connection between the IMD 5 and the electrode(s) 22, 24 through the electrode terminal(s) 32, 34 and a respective conductor present in the lead body 4 of the implantable medical lead 1.

The implantable medical lead 1 typically comprises a respective electrode terminal 32, 34 for each electrode 22, 24 in connection with the distal end 2.

The implantable medical lead 1 also comprises the above-mentioned lead body 4 running from the proximal end 3 to the distal end 2. This lead body 4 comprises an insulating tubing having a channel configured to house the at least one conductor electrically interconnecting the electrode(s) 22, 24 and the electrode terminal(s) 32, 34.

A general aspect of the embodiments relates to an implantable medical lead comprising an insulating tubing having a channel with a distal channel portion and an opposite proximal channel portion. The implantable medical lead also comprises a lead header having a lumen and a fixation helix at least partly present in this lumen of the lead header. A shaft of the implantable medical lead is at least partly present in a lumen of a bearing. The shaft has a first end that is mechanically and typically electrically connected to a first end of the fixation helix and a second end that is electrically connected to a conductor that is at least partly present in the channel of the insulating tubing. The shaft and the fixation helix can thereby be rotated relative the bearing, the lead header and the insulating tubing. Such a rotation of the shaft and fixation helix is translated into a longitudinal movement of the fixation helix relative the lead header. Hence, by rotating the shaft the fixation helix can be rotated and extended out from the lumen in the lead header or be rotated and retracted into the lumen in the lead header.

The lumen of the lead header thereby provides an opening to the outside through which the fixation helix can be moved. This opening, however, provides a passage for blood into the lumen of the lead header and further into the lumen of the bearing and the channel of the insulating tubing.

According to the embodiments a blood seal is provided in the implantable medical lead to prevent or at least reduce or inhibit blood from entering far into the channel of the insulating tubing. The blood seal is in the form of a clot-inducing structure of at least one thrombogenic material, i.e. the clot-inducing structure consists of or is made of one thrombogenic material or multiple different types of thrombogenic materials. This clot-inducing structure is present in the distal channel portion of the insulating tubing or in the lumen of the bearing. The clot-inducing structure triggers formation of a blood clot when blood enters the implantable medical lead and reaches the clot-inducing structure. This trigger or induction of clot formation thereby causes the formation of a physical structure, i.e. a blood clot, which thereby inhibits blood from entering further into the channel towards the opposite proximal portion of the insulating tubing.

When blood enters into the implantable medical lead through the opening in connection with the most distal end of the implantable medical lead the blood will pass further into the implantable medical lead until it comes into contact with the clot-inducing structure. At this point the thrombogenic material of the clot-inducing structure induces the formation of a thrombus or clot at the relevant site in the implantable medical lead. The formed blood clot will physically obstruct the lumen of the bearing or the distal channel portion to thereby block the blood from passing the blood clot.

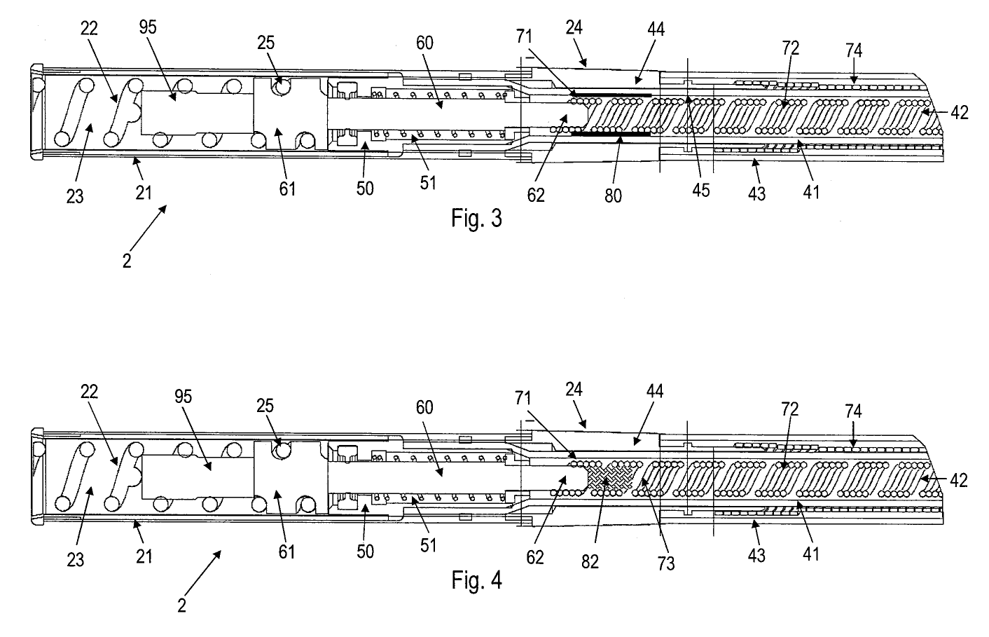

Embodiments will now be further described herein in connection with the drawings starting with FIGS. 3-9. These FIGS. 3-9 illustrate an implantable medical lead in the form of a bipolar lead. As previously mentioned herein, this should merely be seen as an illustrative example and the embodiments are not limited thereto. Hence the relevant feature of the embodiments is the presence of a clot-inducing structure and not the particular organization of the lead components in connection with the distal end of the implantable medical lead. Hence, for other lead types some of the lead components illustrated in FIGS. 3-9 could be omitted or replaced by other lead components selected based on the particular lead type.

FIG. 3 is a cross-sectional view of the distal end 2 of a bipolar implantable medical lead. The implantable medical lead comprises a lead header 21 having a lumen 23 in which a fixation helix 22 is at least partly present. The fixation helix 22 has a first end 25 mechanically and typically electrically connected to a first end 61 of a shaft 60. Various embodiments of interconnecting the fixation helix 22 and the shaft 60 are possible, such as welding or using threads on an outer surface of the first end 61 of the shaft 60 as illustrated in FIG. 3. FIG. 3 also indicates a steroid plug 95 that can be present in the lumen of the fixation helix 22 to reduce the amount of connective tissue deposited around the fixation helix 22 following implantation of the implantable medical lead 1, which is well known in the art.

The shaft 60 is at least partly present in a lumen 51 of a bearing 50, also sometimes denoted coupling. The bearing 50 is in this embodiment mechanically connected to the lead header 21 and provides a connecting bridge or linker between the header 21 and an inner insulating tubing 41. The fixation helix 22 and the shaft 60 are rotatable relative the bearing 50 and also relative the lead header 21. Hence, the shaft 60 and the bearing 50 can in this way be regarded as a rotor and a stator, respectively. A second opposite end 62 of the shaft 60 is electrically connected to a conductor 72, represented by an inner conductor coil 72 in FIG. 3. This means that an electrical connection is achieved from the conductor coil 72 via the shaft 60 and to the fixation helix 22, which can then operate as a pacing and sensing electrode.

The conductor coil 72 is at least partly present in a channel 42 of the inner insulating tubing 41. This inner insulating tubing 41 is arranged between the (inner) conductor coil 72 and an outer conductor coil 74 to thereby electrically insulate the two conductor coils 72, 74 from each other. The outer conductor coil 74 is electrically connected to a ring electrode 24 of the implantable medical lead. An outer insulating tubing 43 is provided outside of the outer conductor coil 74 to provide an outer, insulating layer for the implantable medical lead.

As is seen from FIG. 3, there is an opening at the most distal end of the implantable medical lead through which the fixation helix 22 can be moved. Blood can thereby enter into the lumen 23 of the lead header 21 passing the fixation helix 22 and into the lumen 51 of the bearing 50 and even further into the channel 42 of the inner insulating tubing 41. This channel 42 runs all the way up to the proximal end of the implantable medical lead (see FIG. 2).

In the embodiment illustrated in FIG. 3, a clot-inducing structure 80 is present in a distal channel portion 44 in the interface between an inner surface 45 of the inner insulating tubing 41 and an outer surface of the conductor coil 72. The distal channel portion 44 corresponds in a general embodiment to the half of the inner insulating tubing 41 that is opposite to the end of the inner insulating tubing 41 facing the IMD to which the implantable medical lead is connectable. Thus, the clot-inducing structure 80 can in this general embodiment be positioned anywhere in the inner insulating tubing 41 up to about half the length of the inner insulating tubing 41. In preferred embodiments, the distal channel portion 44 corresponds to a portion of this distal half of the inner insulating tubing 41. The distal channel portion 44 could then correspond to up to about 10-15 cm of the length of the inner insulating tubing 41 if the total length of the inner insulating tubing 41 is about 40-50 cm. It is generally preferred to arrange the clot-inducing structure 80 as close to the most distal end of the inner insulating tubing 41 as physically possible to thereby restrict blood from entering into the channel 42 of the inner insulating tubing 41.

In an embodiment, the clot-inducing structure 80 is in this embodiment in the form of a coating 80 or thin layer of the at least one thrombogenic material applied to the inner surface 45 of the insulating tubing 41 in at least a portion of the distal channel portion 44.

When blood enters the distal channel portion 44 via the lumen 51 of the bearing 50 the blood will, upon contact with the thrombogenic material, start to coagulate to form a blood clot that effectively obstructs the narrow passage between the inner insulating tubing 41 and the conductor coil 72. Hence, the blood is prevented from reaching further into the channel 42 and the implantable medical lead.

The coating 80 applied to the inner surface 45 of the inner insulating tubing 41 could, for instance, be applied to cover a width of about 5 to 15 mm of the inner surface 45. The coating 80 is preferably in the form of a cylinder or ring to cover the full turn of the inner surface 45. Although it is generally preferred to have a coating 80 that covers the full turn of the inner surface 45, the embodiments are not limited thereto. The coating 80 could then instead be in the form of a C-shape, thereby not covering the full turn, as long as sufficient amount of thrombogenic material is present and a sufficient portion of the turn of the inner surface 45 is covered to trigger the formation of a blood clot that is able to restrict further blood flow pass the coating 80 and into the channel 42 of the inner insulating tubing 41. The coating 80 could be a single continuous coating 80 or consist of multiple smaller portions, such as dots, of the at least one thrombogenic material.

FIG. 4 is a cross-sectional view of another embodiment of the distal end 2 of a bipolar lead having a clot-inducing structure 82 that is present in the distal channel portion 44. The clot-inducing structure 82 is in this embodiment in the form of a mesh 82 of the at least one thrombogenic material present in a lumen 73 of the conductor coil 72. The thrombogenic material is in this embodiment preferably a polymer or protein material, which is further discussed herein, to form a mesh 82 or polymer structure similar to a plug present in the most distal part of the lumen 73 of the conductor coil 72.

When blood enters the distal channel portion 44 the blood will come into contact with the thrombogenic material through neighboring turns or loops in the conductor coil 72. There the blood will come into contact with the thrombogenic material triggering a clot forming cascade that will cause the formation of a blood clot in the lumen 73 of the conductor coil 72 and extending between the narrow passage between the conductor coil 72 and the inner insulating tubing 41. The formed clot will thereby effectively inhibit blood from entering further into the channel 42 and the implantable medical lead.

The mesh 82 could be designed to extend about 5 to 15 mm along the lumen 73 of the conductor coil 72.

The embodiments disclosed in FIGS. 3 and 4 can be combined. Thus, the implantable medical lead then comprises a coating 80 of at least one thrombogenic material applied to the inner surface 45 of the inner insulating tubing 41 and a mesh 82 of at least one thrombogenic material present in the lumen 73 of the conductor coil 72.

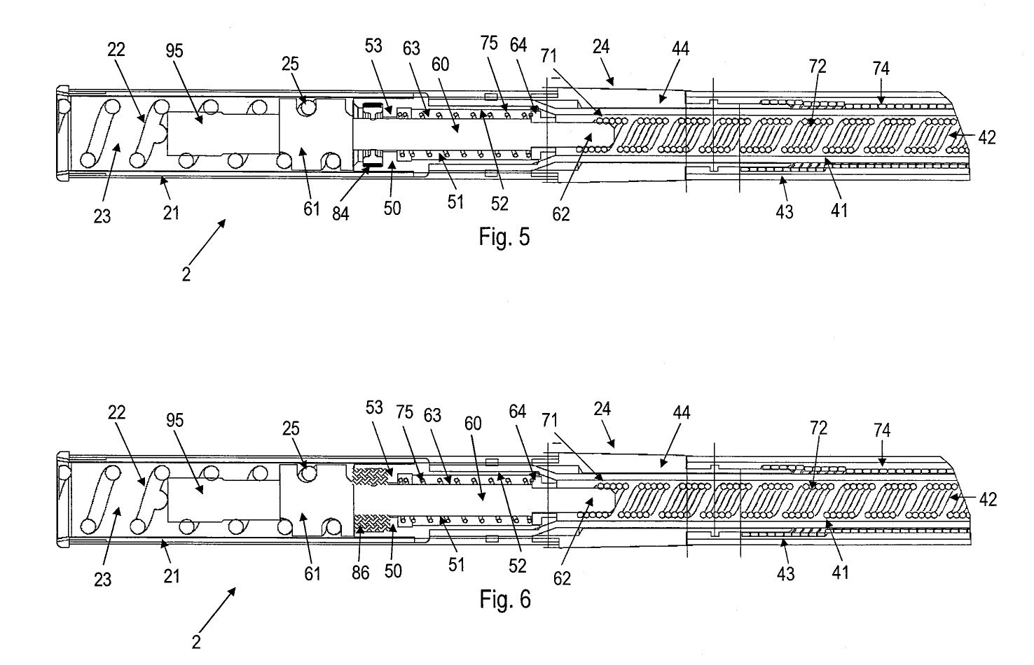

In the embodiments discussed above and disclosed in FIGS. 3 and 4 the clot-inducing structure is present in the distal channel portion 44 of the channel 42 defined by the insulating tubing 41. FIG. 5 illustrates another embodiment with the clot-inducing structure 84 present in the lumen 51 of the bearing 50. The clot-inducing structure 84 is in this embodiment in the form of a coating 84 or thin layer of the at least one thrombogenic material applied to at least a portion of the inner surface 52 of the bearing 50.

In a particular embodiment, the coating 84 of thrombogenic material is preferably provided on the inner surface 52 of a distal part of the bearing 50, i.e. the part of the bearing 50 facing the fixation helix 22. The coating 84 will then trigger formation of a blood clot when blood enters into the lumen 51 of the bearing 50. The formed blood clot will constitute an obstruction to the blood and therefore stop or at least reduce the amount of blood that can pass through the passage between the inner surface 52 of the bearing 50 and the shaft 60.

In similarity to the embodiments disclosed in FIGS. 3 and 4, the coating 84 in FIG. 5 can be replaced by or complemented with a mesh 86 of the at least one thrombogenic material as disclosed in FIG. 6. In this embodiment, the mesh 86 is provided in the interface between an outer surface 63 of the shaft 60 and the inner surface 52 of the bearing 50. For instance, the mesh 86 could be present in a notch or groove in the inner surface 52, i.e. the groove in which the coating 84 of FIG. 5 is provided. Alternatively, the mesh 86 could be attached to the outer surface 63 of the shaft 60, such as in a groove or notch (not illustrated) in the outer surface 63.

Blood entering the implantable medical lead and reaching the lumen 51 of the bearing 50 will come into contact with the thrombogenic material of the mesh 86 and thereby start to coagulate to form a blood clot. The blood clot will block passage of blood further into the lumen 51 and the channel 42 of the inner insulating tubing 41.

For example, the coating 84 and the mesh 86 could be designed to have sizes corresponding to the above mentioned sizes for the coating 80 and the mesh 82.

In another embodiment, the mesh 86 is replaced by a coating of the at least one thrombogenic material applied to at least a portion of the outer surface 63 of the shaft 60.

In an embodiment, the coating 84 of FIG. 5 and the mesh 86 of FIG. 6 can be combined to have two clot-inducing structures present in the lumen 51 of the bearing 50. Alternatively, the coating of FIG. 5 can be combined with a coating applied to at least a portion of the outer surface 63 of the shaft 60.

As is shown in FIGS. 5 and 6, the implantable medical lead could comprise a spring 75 present around the shaft 60 in the lumen 51 of the bearing 50. This spring 75 is provided between a shoulder 64 arranged on the outer surface 63 of the shaft 60 and a shoulder 53 of the bearing 50 extending into the lumen 51. This spring 75 keeps the fixation helix 22 in a retracted position with the sharp end of the fixation helix 22 present in the lumen 23 of the lead header 21. Thus, the spring 75 prevents the fixation helix 22 from unintentionally moving out from the lead header 21, for instance, in connection with implantation where the sharp point could damage tissue during the passage in the subject body.

The coating 84 of FIG. 5 and/or the mesh 86 of FIG. 6 is preferably present distal relative to the spring 75 so that the blood clot induced by the coating 84 and/or mesh 86 will not interfere with the operation of the spring 75. Hence, in a preferred embodiment the blood is caused to clot outside of the spring 75. The shoulder 53 of the bearing 50 typically provides an effective stop for the blood clot, thereby restricting the blood clot to be present in the portion of the interface between the bearing 50 and the shaft 60 distal to the spring-containing portion of the interface between the bearing 50 and the shaft 60.

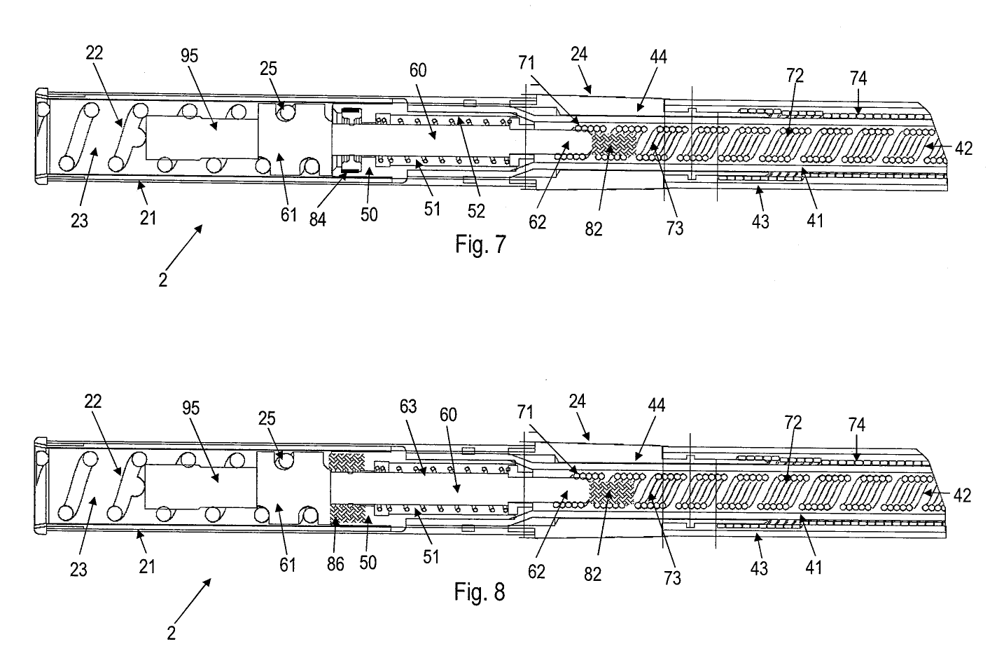

The embodiments disclosed in FIGS. 3 to 6 can be combined as illustrated in FIGS. 7 and 8. Hence, in an embodiment the implantable medical lead comprises a first clot-inducing structure 82 present in the distal channel portion 44. In FIGS. 7 and 8, this first clot-inducing structure 82 is represented by a mesh 82 present in the lumen 73 of the conductor coil 72. Alternatively, the first clot-inducing structure could be a coating applied to the inner surface 45 of the insulating tubing 41 as shown in FIG. 3 or the first clot-inducing structure could be a combination of the mesh 82 and the coating.

The implantable medical lead also comprises a second clot-inducing structure 84, 86 of at least one thrombogenic material present in the lumen 51 of the bearing 50. The second clot-inducing structure 84 could be in the form of a coating 84 of the at least one thrombogenic material arranged on the inner surface 52 of the bearing 50, see FIG. 7. Alternatively, or in addition, the second clot-inducing structure 86 could be in the form of a mesh 86 or a coating of the at least one thrombogenic material applied on the outer surface 63 of the shaft 60, see FIG. 8.

In the embodiments illustrated in FIGS. 2-8, rotation of the fixation helix 22 is typically achieved by rotating a connector pin corresponding to the electrode terminal 32 of FIG. 2. Rotation of the connector pin 32 relative to the lead body 4 causes a rotation of the conductor coil 72 inside the channel 42 of the inner insulating tubing 41. The electrical and mechanical connection between the first end 71 of the conductor coil 72 and the second end 62 of the shaft 60 implies that also the shaft 60 and the attached fixation helix 22 are rotated relative to the lead body 4, the bearing 50 and the lead header 21.

The clot-inducing structure 80, 82, 84, 86 of the embodiments provides an effective sealing function by forming an obstruction that the blood cannot pass. When the blood comes into contact with the thrombogenic material a clot-forming (coagulation) reaction or cascade is initiated leading to a rapid activation and recruitment of cellular (platelet) and protein (coagulation factors) components. Hence, the clot will form rapidly following the first contact with the blood inside the implantable medical lead.

The blood clot formed due to the clot-inducing structure 80, 82, 84, 86 will not interfere with the operation of the implantable medical lead and in particular the extension and retraction of the fixation helix 22. Thus, even though a blood clot forms in the interface between the bearing 50 and the shaft 60 and/or in the interface between the conductor coil 72 and the inner insulating tubing 41, the shaft 60 is still rotatable relative the bearing 50 and the conductor coil 72 is still rotatable relative the inner insulating tubing 41. A reason for this is that the clot-inducing structure 80, 82, 84, 86 does not per se swell when coming in contact with the blood. Hence, its physical integrity remains. The blood clot is instead basically started at the surface of the clot-inducing structure 80, 82, 84, 86 and optionally inside the clot-inducing structure 82, 86 when provided as a porous mesh 82, 86. The clot will then form on the surface of the clot-inducing structure 80, 82, 84, 86 and extend into the above-mentioned interface(s). However, the clot will generally not grip so tightly in the opposite structure, i.e. conductor coil 72 in FIG. 3, inner insulating tubing 41 in FIG. 4, shaft 60 in FIG. 5 and bearing 50 in FIG. 6 that rotation of the conductor coil 72, the shaft 60 and the fixation helix 22 is prevented.

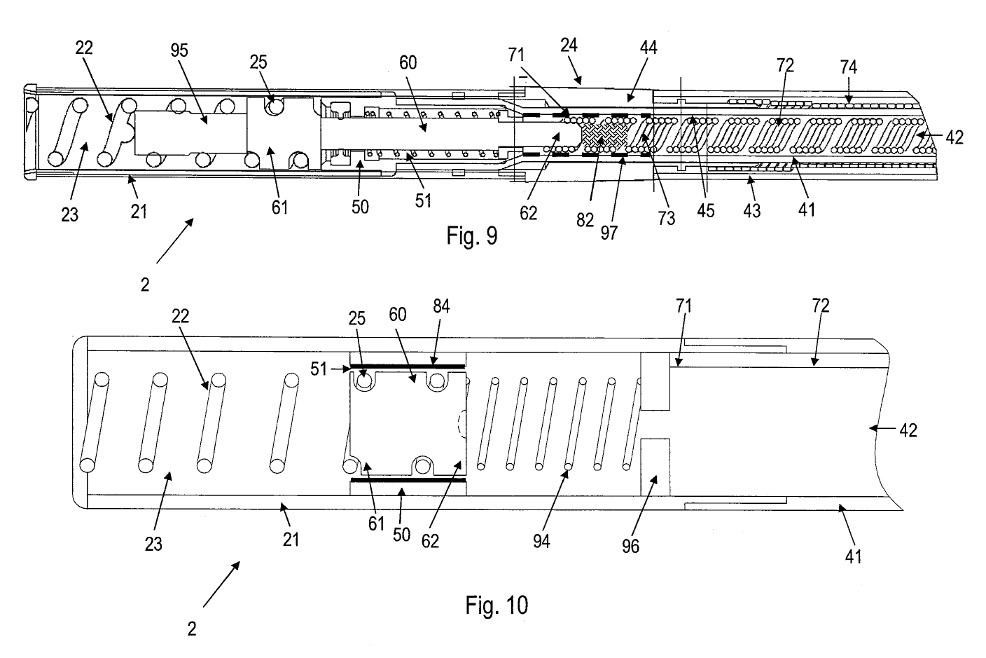

In order to further reduce the risk of the clot locking the above-mentioned rotational movement, the opposite surface of the interface relative the clot-inducing structure 80, 82, 84, 86 can be coated with or provided with a lubricant layer or coating 97 as illustrated in FIG. 9. In this embodiment, the clot-inducing structure 82 is in the form of a mesh 82 provided in the lumen 73 of the conductor coil 72. The opposite surface that the clot will engage will be the inner surface 45 of the inner insulating tubing 41. At least a portion of this surface 45 in the distal channel portion 44 can thereby be provided with a lubricant layer 97 that prevents the formed blood clot from tightly attach to and grip the inner insulating tubing 41. In FIG. 9 this lubricant layer 97 has been indicted with broken lines. When rotating the conductor coil 72 typically also the mesh 82 and the formed blood clot attached to the mesh 82 will rotate relative the lubricant layer 97 and the inner insulating tubing 41.

A corresponding lubricant layer could also or instead be provided in connection with other embodiments of the clot-inducing structure 80, 82, 84, 86 as illustrated in FIGS. 3-8.

The lubricant layer can be of and consist of any material that prevents or at least reduces the risk of the blood clot from tightly becoming attached to the lubricant layer. Non-limited examples of such materials include polyvinylpyrrolidone (PVP) and phosphorylcholine.

A guide wire or stylet is generally introduced into the lumen of the inner conductor coil during implantation to guide the implantable medical lead to the intended implantation site in the subject body. Stylet jam in the lumen during implantation is a severe complication since the implantable medical lead might then have to be replaced. Such stylet jam can be due to blood leakage into the lumen. If the blood leaks into the lumen while the stylet is inserted, coagulation of the blood may fixate the stylet to the implantable medical lead. Correspondingly, if the blood leaks into the lumen prior to insertion of the stylet, a blood clot can be formed inside the lumen preventing any attempt to insert the stylet.

The clot-inducing structures of the embodiments effectively reduce the risk of such stylet jams.

If the clot-inducing structure is in the form of a mesh present in the lumen of the inner conductor coil, the stylet could be prevented from entering the mesh either by designing the stylet so that its end will not reach into the mesh or by using a cage. An example of such a cage 100 is illustrated in FIG. 12. The cage 100 is then dimensioned to be present in the lumen of the conductor coil and comprises the mesh 82 of the at least one thrombogenic material. The cage 100 is generally in the form of a cylinder having a first end surface 120 facing the shaft when present in the lumen. A second, opposite end surface 110 is closed as illustrated in FIG. 12. The lateral surface 130 of the cage 100 comprises at least one opening 140 to allow blood to enter into the cage 100 and come into contact with the mesh 82. Such openings 140 may optionally also be present in the first end surface 120 of the cage 100. In a preferred embodiment, the lateral surface 130 comprises multiple openings 140 and can be perforated or is in the form of a net to enable blood to come into contact with the mesh and the thrombogenic material. However, the cage 100 still has sufficient integrity so that it will not collapse when pressure is applied to the first and second end surfaces 110, 120. The cage 100 could therefore be manufactured by various metal or hard plastic materials.

When a stylet is introduced in the lumen of the inner conductor coil the end of the stylet will come into contact with the second end surface 120 of the cage 100 that is closed. This second end surface 120 and the cage 100 thereby functions as a mechanical stop preventing the stylet from entering the mesh 82.

FIG. 10 is a cross-sectional view of the distal end 2 of another type of implantable medical lead. In this embodiment, the shaft 60 functions as a support for the fixation helix 22 and is present in the lumen 51 of the bearing 50. The conductor 72 that is electrically connected to the fixation helix 22 is in this embodiment not necessarily a conductor coil but rather a conductive wire 72. The conductive wire 72 has a first end 71 electrically connected to a shoulder structure 96 that is in the form of a ring or disc with a central opening provided on the inner surface of the header 21. This shoulder structure 96 is typically of a metal material and is electrically conductive. A conductive spring 94 is attached to the shoulder structure 96 and to the second end 62 of the shaft 60. This conductive spring 94 basically operates similar to the spring discussed in connection with FIG. 5 but in addition provides an electrical connection between the shaft 60 and the shoulder structure 96 and thereby between the fixation helix 22 and the conductive wire 72.

Helix extension and retraction is, in this embodiment, achieved by a stylet introduced into the lumen 42 of the insulating tubing 41, which is attached (directly or indirectly) to the lead header 21. The stylet enters the central opening in the shoulder structure 96 and reaches an indentation or notch in the shaft 60 (schematically indicated with broken lines in FIG. 10). The stylet could then operate as a screw driver to rotate the shaft 60 and the fixation helix 22.

In the embodiment illustrated in FIG. 10 and in the previous embodiments of FIGS. 3-9, a post or other structure (not illustrated) protruding from the inner surface of the lead header 21 between adjacent turns of the fixation helix 22 will translate a rotation of the fixation helix 22 into a longitudinal movement of the fixation helix 22, which is well known in the art.

In FIG. 10 a clot-inducing structure 84 of at least one thrombogenic material is provided in the lumen 51 of the bearing 50. The clot-inducing structure 84 is preferably in the form of a coating of the at least one thrombogenic material applied to the inner surface of the bearing 50 and/or to the outer surface of the shaft 60.

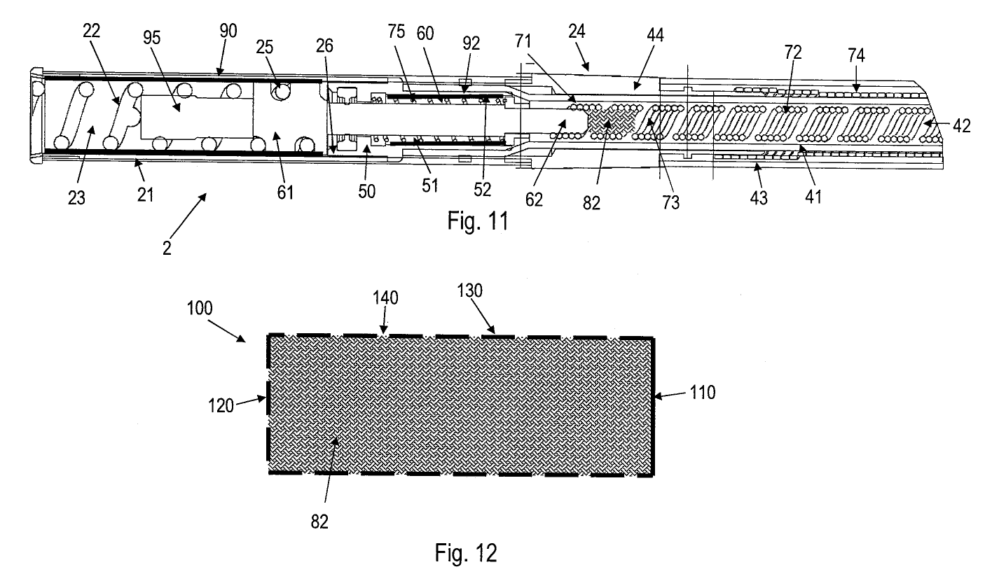

Generally, clot formation should be prevented in connection with the fixation helix of the implantable medical lead and in connection with the optional (conductive) spring. Such undesired clot formation at those parts of the implantable medical lead can be prevented or at least reduced by using anticoagulant coatings. FIG. 11 illustrates this concept. Thus, an anticoagulant coating 90 of an anticoagulant material can be provided on the inner surface 26 of the lead header 21 to inhibit blood clotting at the fixation helix 22 when blood enters the lumen 23 of the lead header 21. Alternatively, or in addition an anticoagulant coating 92 of an anticoagulant material can be provided on the inner surface 52 of the bearing 50 to inhibit blood clotting in connection with the spring 75 when blood enters the lumen 51 of the bearing 50.

Thus, in particular embodiments blood clotting is induced at one or multiple particular sites inside the implantable medical lead to form a blood clot that functions as a biological blood seal. However, blood clotting is preferably also inhibited at one or more other sites inside the implantable medical lead where such blood clots could interfere with the extension and retraction of the fixation helix 22.

The anticoagulant material could be any non-toxic implantable anticoagulant material that reduces the risk of local clotting in connection with the anticoagulant coating(s) 90, 92. A non-limiting example of such an anticoagulant material that can be used according to the embodiments is heparin. Heparin coatings of metals and polymers are widely known in the medical device and implant industry. An example of such commercially available heparin coatings is available from Corline.

An example of a thrombogenic material that can be used according to the embodiments is collagen. Collagen is an endogenous thrombogenic protein that induces formation of a thrombus when contacting blood. Other examples of thrombogenic materials that can be used according to the embodiments include silicon or glass. Further examples include hydrophobic materials and in particular hydrophobic polymers.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. An implantable medical lead comprising:
an insulating tubing having a channel with a distal channel portion and an opposite proximal portion;
a lead header having a lumen;
a bearing having a lumen;
a fixation helix at least partly present in the lumen of the lead header and having a first end;
a shaft at least partly present in the lumen of the bearing and having a first end mechanically connected to the first end of the fixation helix and a second end; and
a conductor at least partly present in the channel and having a first end electrically connected to the second end of the shaft, wherein a first clot-inducing structure of at least one thrombogenic material is present i) in the distal channel portion or ii) in the lumen of the bearing to trigger formation of a blood clot when blood enters the implantable medical lead and thereby inhibit blood from entering further into the channel towards the opposite proximal portion.

2. The implantable medical lead according to claim 1, wherein the first clot-inducing structure is present in the lumen of the bearing.

3. The implantable medical lead according to claim 2, wherein the first clot-inducing structure is arranged on an outer surface of the shaft.

4. The implantable medical lead according to claim 2, wherein the first clot-inducing structure is arranged on an inner surface of the bearing.

5. The implantable medical lead according to claim 1, wherein the first clot-inducing structure is present in the distal channel portion.

6. The implantable medical lead according to claim 5, wherein the first clot-inducing structure is a coating of the at least one thrombogenic material applied to an inner surface of the insulating tubing in at least a portion of the distal channel portion.

7. The implantable medical lead according to claim 5, wherein
the conductor is a conductor coil having a first end electrically and mechanically connected to the second end of the shaft; and
the first clot-inducing structure is a mesh of the at least one thrombogenic material present in a lumen of the conductor coil.

8. The implantable medical lead according to claim 6, wherein a lubricant layer is arranged on an inner surface of the insulating tubing in at least a portion of the distal channel portion.

9. The implantable medical lead according to claim 7, wherein a cage is present in the lumen of the conductor coil and wherein the cage has the mesh of the at least one thrombogenic material, wherein the cage is in the form of a cylinder having a first end surface facing the shaft, a second opposite end surface that is closed and a lateral surface having at least one opening to allow blood to enter into the cage and come into contact with the mesh of the at least one thrombogenic material.

10. The implantable medical lead according to claim 5, wherein a second clot-inducing structure of at least one thrombogenic material is present in the lumen of the bearing.

11. The implantable medical lead according to claim 10, wherein the second clot-inducing structure is arranged on an outer surface of the shaft.

12. The implantable medical lead according to claim 10, wherein the second clot-inducing structure is arranged on an inner surface of the bearing.

13. The implantable medical lead according to claim 1, wherein an anticoagulant coating of an anticoagulant material is provided on an inner surface of the lead header to inhibit blood clotting when blood enters the lumen of the lead header.

14. The implantable medical lead according to claim 1, wherein an anticoagulant coating of an anticoagulant material is provided on an inner surface of the bearing to inhibit blood clotting when blood enters the lumen of the bearing.

15. The implantable medical lead according to claim 13, wherein the anticoagulant material is heparin.

16. The implantable medical lead according to claim 1, wherein the thrombogenic material is collagen.
